(19) United States
(12) Patent Application Publication
(10) Pub. No.: US 2024/0085328 A1
(43) Pub. Date: Mar. 14, 2024

(54) EXCITATION SPECTRAL MICROSCOPY FOR HIGHLY MULTIPLEXED FLUORESCENCE IMAGING AND QUANTITATIVE BIOSENSING

(71) Applicant: THE REGENTS OF THE UNIVERSITY OF CALIFORNIA, Oakland, CA (US)

(72) Inventors: Ke Xu, Kensington, CA (US); Kun Chen, El Cerrito, CA (US); Rui Yan, Berkeley, CA (US); Limin Xiang, El Cerrito, CA (US)

(73) Assignee: THE REGENTS OF THE UNIVERSITY OF CALIFORNIA, Oakland, CA (US)

(21) Appl. No.: 18/484,951

(22) Filed: Oct. 11, 2023

Related U.S. Application Data
(63) Continuation of application No. PCT/US2022/024625, filed on Apr. 13, 2022.
(60) Provisional application No. 63/174,070, filed on Apr. 13, 2021.

(51) Int. Cl. G01N 21/64; G01N 33/58

(52) U.S. Cl. CPC G01N 21/6428; G01N 21/6458; G01N 33/582; G01N 2021/6439; G01N 2201/067; G01N 2201/0683

(57) ABSTRACT

Systems and methods are provided for excitation spectral microscopy using frame-synchronized acousto-optic scanning of fluorescent excitation wavelengths. Linear unmixing of the images of targets of components that are individually labeled with different fluorophores can be simultaneously imaged with high temporal resolution and low crosstalk and the local abundance of each fluorophore at each pixel can be quantified. Fluorophore decomposed micrographs of the sample can be obtained by rendering the abundance in each pixel as an image.

### CROSS-REFERENCE TO RELATED APPLICATIONS

This application claims priority to, and is a 35 U.S.C. § 111(a) continuation of, PCT international application number PCT/US2022/024625 filed on Apr. 13, 2022, incorporated herein by reference in its entirety, which claims priority to, and the benefit of, U.S. provisional patent application Ser. No. 63/174,070 filed on Apr. 13, 2021, incorporated herein by reference in its entirety. Priority is claimed to each of the foregoing applications.

The above-referenced PCT international application was published as PCT International Publication No. WO 2022/221414 A1 on Oct. 20, 2022, which publication is incorporated herein by reference in its entirety.

### STATEMENT REGARDING FEDERALLY SPONSORED RESEARCH OR DEVELOPMENT

This invention was made with government support under GM132681 awarded by the National Institutes of Health. The government has certain rights in the invention.

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to systems and methods for fluorescence microscopy and imaging and more particularly to fast and highly multiplexed systems and methods for simultaneous imaging of multiple fluorophores labeled subcellular targets with low crosstalk and high temporal resolution. The system and methods use frame-synchronized fast scanning of the excitation wavelength from a white lamp via an acousto-optic tunable filter (AOTF) and a single, fixed fluorescence emission detection band. The methods can quantify the abundances of different fluorophores in the same sample through unmixing of the excitation spectra.

### 2. Background

Target specificity and compatibility with live cells are two distinct advantages provided by fluorescence microscopy in biological research, However, the benefits of fluorescence microscopy come with some performance limits. Due to the broad spectral width of molecular fluorescence, common approaches based on bandpass filters can only accommodate 3 or 4 spectrally well-separated channels within the visible range.

Attempting to image fast dynamics in live cell with multi-target imaging is often impeded by the slow mechanical switching between filter sets. Simultaneous monitoring of two or more spectral channels is often necessary with quantitative imaging of fluorescent biosensors and creates yet another challenge for fast imaging and its integration with additional fluorescent markers is even more difficult.

Spectral imaging offers potential solutions to these problems. Although it is conceptually intuitive to detect the fluorescence emission spectrum with common pixel array-based cameras, in practice it remains difficult to record spatial and spectral information at the same time for the wide-field. As a consequence, spectrally resolved images are usually acquired in a point-scanning manner by dispersing the fluorescence from one single spot of the sample at a time. Alternatively, sparse single molecules may be spectrally dispersed in the wide-field to accumulate super-resolution images over many camera frames.

Unfortunately, both of these scenarios place substantial constraints on temporal resolution. While tunable bandpass filters may allow one to scan through the emission wavelength in the wide-field, the application of narrow bandpass filtering to the fluorescence emission results is an inefficient use of the scarce signal. In addition, tunable thin-film filters are limited in speed with mechanical scanning, whereas electronically tunable filters, e.g., acousto-optic tunable filter (AOTF), only work for polarized light and introduce distortion into the wide-field images.

In contrast to these challenges to resolve the fluorescence emission spectrum at every pixel, it is straightforward to scan the excitation wavelength for the entire imaging field while recording the resultant emission in a fixed passband to produce excitation wavelength-resolved images. Excitation spectral microscopy, however, is conceptually less intuitive and experimentally rarely explored, especially for examining subcellular structural dynamics in live cells.

Accordingly, there is a need for new devices and schemes for multiplexed fluorescence microscopy for cell and tissue samples and quantitative imaging of biosensors in live cells.

### BRIEF SUMMARY

The multiplexing capability of conventional fluorescence microscopy is severely limited by the broad spectral width of fluorescence and conventional approaches of dispersing the local emission spectra notably impede the attainable throughput. The present technology overcomes these deficiencies by providing systems and methods using a single, fixed fluorescence emission detection band along with frame-synchronized fast scanning of the excitation wavelength from a white lamp via an acousto-optic tunable filter (AOTF). Multiple subcellular fluorophore labeled targets with substantial spectral overlap can be simultaneously imaged in the wide-field with low crosstalk and high spatiotemporal resolutions. In one embodiment, up to six subcellular targets, labeled by common fluorophores of substantial spectral overlap, can be simultaneously imaged in live cells with low (˜1%) crosstalk and high temporal resolutions (down to ˜10 ms).

The demonstrated capability to quantify the abundances of different fluorophores in the same sample through unmixing of the excitation spectra enables the development of novel, fast, quantitative imaging schemes for different modes of fluorescent biosensors in live cells, as well as multiplexing with multiple additional fluorescent tags. In one embodiment, for example, quantitative imaging schemes are provided for both bi-state and Förster resonance energy transfer fluorescent biosensors in live cells.

In another example, the methods and systems achieve high sensitivities and spatiotemporal resolutions in quantifying the mitochondrial matrix pH and intracellular macromolecular crowding, and further demonstrate, for the first time, the multiplexing of absolute pH imaging with three additional target organelles/proteins to elucidate the complex, Parkin-mediated mitophagy pathway.

In one specific example, the pH-dependent abundances of the bi-state sensor pHRed in its protonated and deprotonated states were quantified, and well fitted to the ideal Henderson-Hasselbalch equation to enable absolute pH imaging, thus visualizing concurrent fast changes in the mitochondrial shape and matrix pH.

In another example, a microscopy version of the early excitation spectroscopy approaches to quantify FRET efficiency was established and achieved high sensitivities and spatiotemporal resolutions in unveiling rich dynamics for macromolecular crowding in live cells.

In another illustration, the biosensor and multi-fluorophore imaging capabilities were integrated and achieved absolute pH imaging with three additional fluorophore labels to elucidate the Parkin-mediated mitophagy pathway, thus unveiling fascinating spatiotemporal dynamics for this complex, multistate system.

Accordingly, the described excitation spectral microscopy provides exceptional opportunities for highly multiplexed fluorescence imaging. The prospect of acquiring fast spectral images without the need for fluorescence dispersion or care for the spectral response of the detector offers tremendous potential for adaptation.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a schematic system diagram of one system of excitation spectral microscopy showing polarizer, lens, bandpass filter, and dichroic mirror components according to one embodiment of the technology. Full-frame spectral micrographs can be obtained by the synchronized fast modulation of the excitation wavelength A in consecutive frames.

FIG. 2 is a functional block diagram of a method for excitation spectral microscopy and highly multiplexed imaging according to one embodiment of the technology.

FIG. 3A is a plot of the measured AOTF output (microscope input) spectral profiles for the 8 excitation wavelengths used for the 10 fps fixed-cell demonstration.

FIG. 3B is a plot of the expected recorded excitation spectra of different fluorophores at the 8 excitation wavelengths for the 6-target fixed-cell demonstration obtained by multiplying the power-normalized spectral profiles in FIG. 3A with the literature absorption spectra of the fluorophores.

FIG. 4A is a plot of the measured AOTF output (microscope input) spectral profiles for the 8 excitation wavelengths used for the 10 fps live-cell demonstration.

FIG. 4B is a plot of the expected recorded excitation spectra of different fluorophores at the 8 excitation wavelengths for the 6-target fixed-cell demonstration obtained by multiplying the power-normalized spectral profiles in FIG. 4A with the literature absorption spectra of the fluorophores.

### DETAILED DESCRIPTION

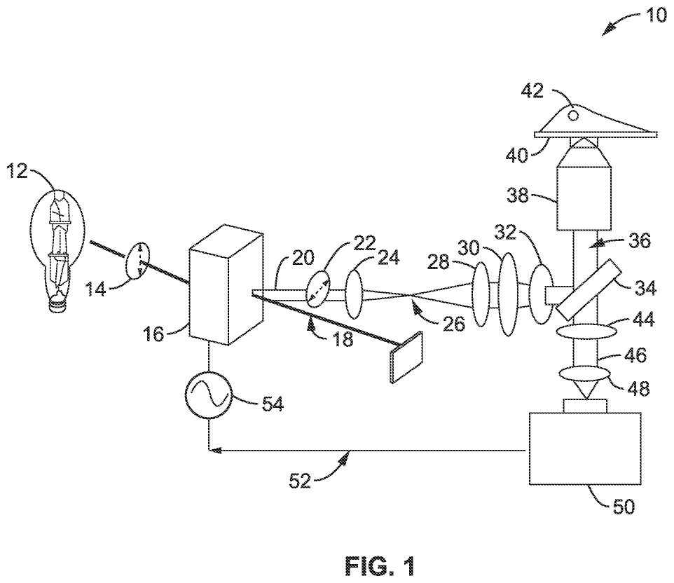

Referring more specifically to the drawings, for illustrative purposes, systems and methods for excitation spectral microscopy with multiplexed florescence imaging are generally shown. Several embodiments of the technology are described generally in FIG. 1 through FIG. 4B to illustrate the characteristics and functionality of the devices, systems and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

Turning now to FIG. 1, one embodiment 10 of an optical set up for fluorescence imaging is shown schematically. Light from a light source 12 such as a white lamp is preferably collimated and linearly polarized with a first polarizer 14 before entering an acousto-optic tunable filter (AOTF) 16.

The AOTF 16 is an electronically tunable filter that can modulate the wavelength and intensity of light from one or more light sources. In this application, the AOTF is used as a tunable filter where the first-order diffracted component 20 is ultimately allowed to illuminate the specimen while the zeroth-order beam 18 is blocked.

The 1st order diffracted beam 20, with its wavelength and intensity controlled by a radio-frequency (RF) synthesizer 54 and polarization orientation rotated by 90°, may be cleaned up with a second polarizer 22 and the resulting beam 26 directed through lens 24, lens 28 and lens 30 and single band filter 32. The lens configuration shown is typical for a commercial epifluorescence microscope. The resulting beam from the filter 32 is used as the excitation source. The excitation source beam 36 is directed to a dichroic mirror 34 and through the objective 38 to the transparent platform 40 to the sample 42 in this illustration.

The fluorescence of the sample 42 is imaged through the objective 38, dichroic mirror 34 and bandpass filter 44. The filtered light 46 is focused with lens 48 to the camera 50. Particularly preferred cameras 50 are sCMOS or EM-CCD cameras. The camera 50 shown schematically in FIG. 1, preferably includes or is coupled with a processor with a non-transitory memory storing instructions that are executable by the processor to acquire, process, render, record and display images as well as control the timing and characteristics of the excitation beam that is produced and impinged on a target sample and the frame rate. In one preferred embodiment, the programming also includes preset wavelength profiles of many different fluorophores. The wavelength profiles are preferably excitation beam characteristics including wavelength ranges, bandwidth, exposure times and intensities that have been optimized for the specific fluorophore label that is selected and used.

Full-frame spectral micrographs are obtained by the synchronized fast modulation of the excitation wavelength A in consecutive frames with this system.

A multifunction I/O device reads out the frame signal of the camera 50 to synchronize the RF synthesizer 54, so that for each successive frame the excitation is switched between preset wavelength profiles with a trigger signal 52. For example, if there are 8 profiles that each have a bandwidth of 5-12 nm and typical intensities between ˜6 μW and ˜27 μW that are recorded at 10 frames per second (fps), then full-frame excitation-spectral images will be produced every 8 camera frames.

With this setup, that uses a standard epifluorescence microscope with a single emission band, multiple subcellular targets that have been labeled with common fluorophores that may have substantial spectral overlap, can be simultaneously imaged in live cells in the wide-field with low crosstalk and high spatiotemporal resolution by using frame-synchronized fast scanning of the excitation wavelength from a white lamp light source.

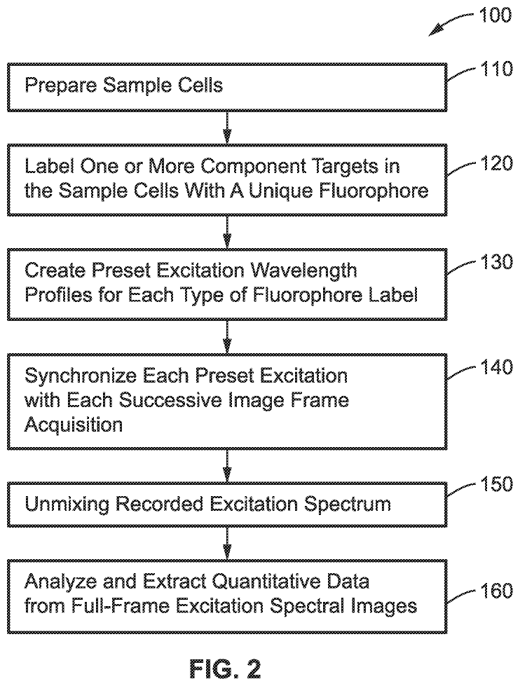

Referring now to FIG. 2, an embodiment of the method 100 for assembling macromolecular structures from cultured cells and scaffolds is shown schematically. At block 110, sample cells are selected and cellular structures or features of interest are selected for evaluation. Any feature or component of the cells or other targets that can be labeled by a fluorochrome can be selected at block 110. Fluorescent dyes or other fluorescent labeling vehicles are also selected for optimal use on each selected feature.

The selected fluorescent labels are applied to the sample cells to label one or more selected component targets. Each target cellular component or feature is labeled with a selected i.e. unique fluorophore at block 120.

At block 130 of FIG. 2, a pre-calibrated excitation profile is created for each type of fluorophore label that is selected. If a preset wavelength excitation profile for a particular fluorophore has already been created, then the existing profile may be selected at block 130. The excitation spectrum is a range of light wavelengths that add energy to a particular fluorophore, causing it to emit wavelengths of light called the emission spectrum. The excitation profile that is created for each fluorophore at block 130 preferably includes an optimized narrow range of wavelengths, a bandwidth range and an intensity range. Optimum times of exposure may also be included in the profile. In one embodiment, reference wavelength excitation spectra of each fluorophore are separately measured on the setup using singly labeled samples and characteristics optimized to create each excitation profile.

Imaging of the fluorescence of the sample over time at block 140 is synchronized with sequential excitation by each of the selected preset excitation profiles. The source excitation of the sample is preferably switched between each of the preset excitation profiles for each successive frame acquisition. In this way, full frame excitation spectral images can be obtained every complete cycle of the selected preset excitation profiles (i.e. the number of applied fluorophore labels).

The recorded excitation spectral images that are acquired are then unmixed at block 150 of the method of FIG. 2. Decomposed images of the each of the selected fluorophores are obtained via linearly unmixing of the excitation-dependent intensity at each pixel in the image using the reference spectra of the profiles.

Since each fluorophore is characterized by its own excitation spectrum, i.e., a fixed profile of how the fluorescence intensity varies as a function of the excitation wavelength, a sample region containing multiple fluorophores will exhibit an excitation spectrum that is a linear combination of that of its component fluorophores. Thus, by linearly unmixing the recorded excitation spectrum of every pixel based on the excitation spectrum of each fluorophore pre-calibrated on the same setup using singly labeled samples, it is possible to quantify the local abundance of each fluorophore. Rendering the resultant abundance in each pixel as an image will yield fluorophore-decomposed micrographs of the sample.

Finally, the individual or sequence of full frame images are further analyzed, and quantitative data extracted at block 160.

It can be seen that by synchronizing the excitation wavelength to each camera frame through fast electronic modulation of the AOTF, full-frame excitation spectral images can be obtained with no moving parts at high temporal resolution that are effectively only limited by the camera framerate and the desired number of excitation wavelengths.

As an additional benefit, as fluorescence detection can be performed for a fixed emission band, the measured spectral behaviors are robustly defined by the preset excitation profiles and independent of the spectral response of the detection system.

The ability to unmix and quantify different, spectrally overlapped fluorescent species in the same sample via the excitation spectrum can be adapted to create fast, quantitative imaging schemes for fluorescent biosensors in live cells, as well as their multiplexing with additional fluorophores.

Although the apparatus and methods used to illustrate certain aspects of the technology were generally based on a lamp-operated epifluorescence microscope, it will be understood that other adaptions are possible. For example, the fast multi-fluorophore and quantitative biosensor imaging capabilities described here should be readily extendable to other systems, including light-sheet fluorescence microscopy and structured illumination microscopy. In these adaptations, frame-synchronized fast wavelength scanning may be implemented with supercontinuum light sources. The prospect of acquiring fast spectral images in the wide-field without the need for fluorescence dispersion or the care for the spectral response of the detector offers tremendous potential utilizations.

The technology described herein may be better understood with reference to the accompanying examples, which are intended for purposes of illustration only and should not be construed as in any sense limiting the scope of the technology described herein as defined in the claims appended hereto.

### Example 1

In order to demonstrate the functionality of the system and methods, one embodiment of the optical imaging system was constructed and basic methods tested. White light from a plasma lamp (HPLS301, Thorlabs) was collimated into a beam about 4 mm in diameter, and linearly polarized through a wire grid polarizer (WP25M-VIS, Thorlabs). The polarized beam entered an acousto-optic tunable filter (AOTF) (EFLF100L1, Panasonic), and the 1st order diffracted beam, with its polarization direction rotated by 90° versus the incident beam, was cleaned up with a second polarizer (WP25M-VIS) mounted perpendicular to the first polarizer. This excitation beam was coupled into an Olympus IX73 inverted epifluorescence microscope and focused at the back focal plane of an oil-immersion objective lens (Olympus, UPLSAPO100XO, NA 1.40), thus illuminating a sample area of approximately 70 μm diameter. The excitation filter, dichroic mirror, and emission filter that were used were FF01-505/119, FF573-Di01, and FF01-630/92, respectively, from Semrock.

The AOTF unit was driven by an 8-channel RF synthesizer (97-03926-12, Gooch & Housego). The excitation wavelength profiles at different applied RF frequencies were measured using a visible spectrometer (USB4000-VIS-NIR, Ocean Optics), with power levels determined by a photodiode power sensor (S120VC, Thorlabs). Typical excitation power was about 6 μW [for recording at 10 frames per second (fps)] and about 27 μW (for recording at 240 and 202 fps) at each wavelength, corresponding to a power density of approximately 0.16-0.72 W/cm² at the sample, which allowed adequate signal and minimal photobleaching.

Prolonged recordings were performed with lowered excitation powers of about 2 μW. Wide-field fluorescence images were continuously recorded using an sCMOS camera (Zyla 4.2, Andor) at an effective pixel size of 130 nm (after 2×2 binning) for 512×512 pixels (67×67 μm2 field of view) at 10 fps or for 512×150 pixels (67×20 μm² field of view) at 202 fps, or an EMCCD camera (iXon Ultra 897, Andor) at an effective pixel size of 160 nm for 448×110 pixels (72×18 μm² field of view) at 240 fps. The camera was internally triggered, and the “fire” (exposure) output trigger signal was read by a multifunction I/O board (PCI-6733, National Instruments), which in turn modulated the RF synthesizer for the synchronized control of the excitation wavelength in each camera frame.

### Example 2

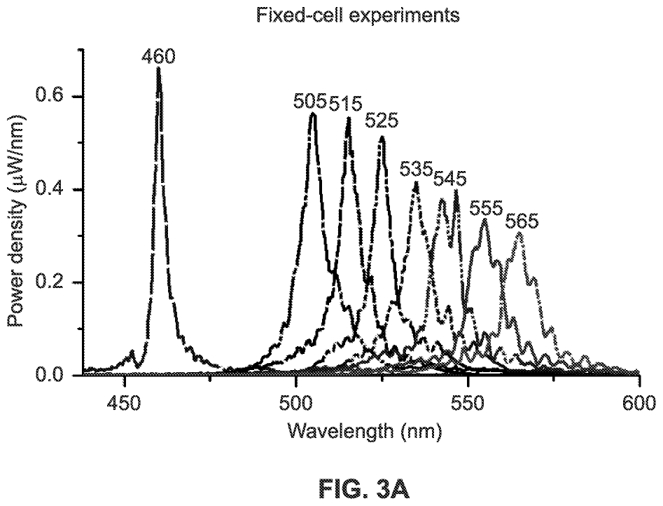

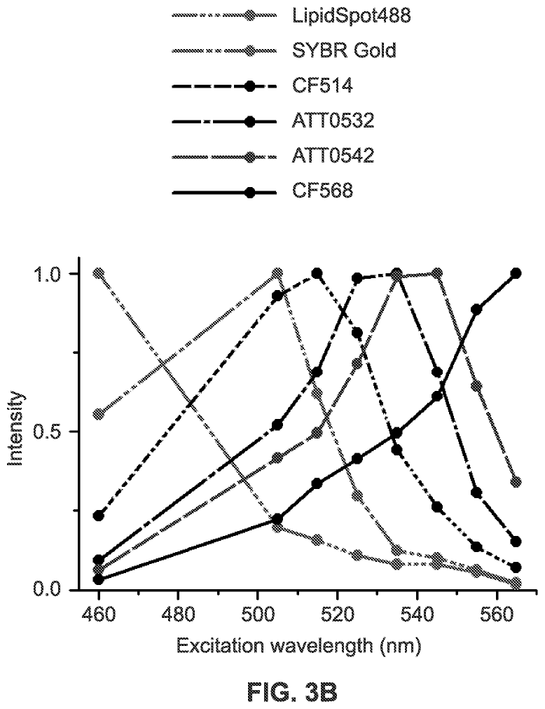

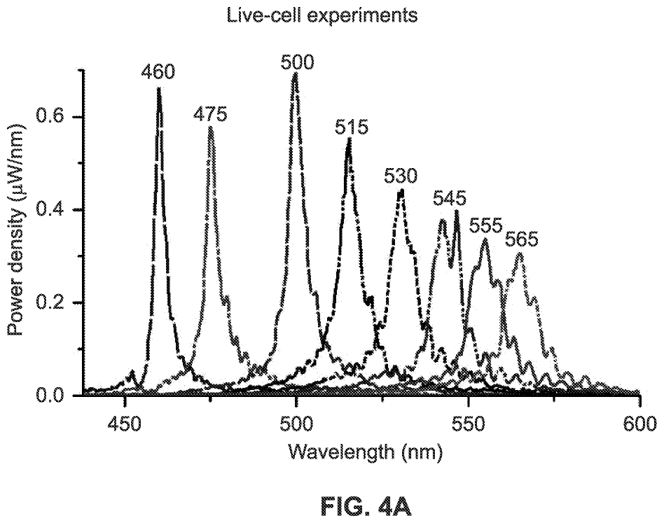

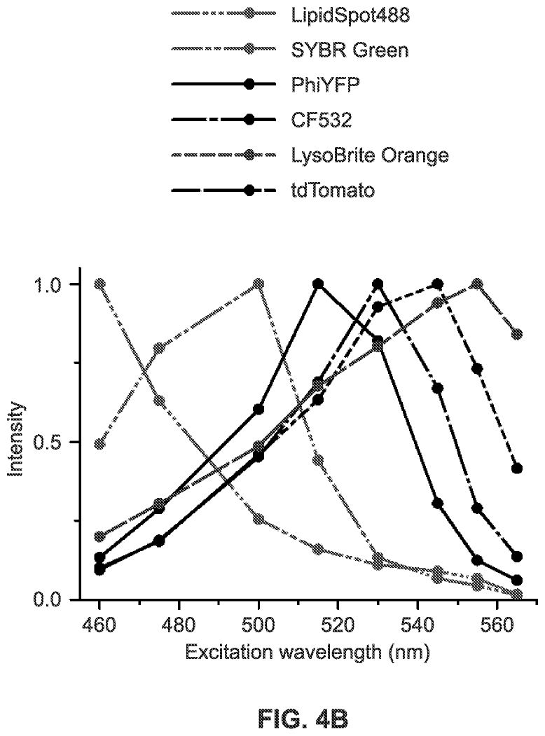

In order to demonstrate the functionality of the detection methods, fast multitarget imaging of fixed cells and live cells was performed using the basic imaging system of Example 1. Initially, the reference excitation spectrum of each of the selected fluorophores was obtained from singly labeled live and fixed cells by recording images at eight preset excitation wavelength profiles. Measured AOTF output (i.e. microscope input) of spectral profiles for the 8 excitation wavelengths used for the 10 fps fixed-cell demonstration are shown in FIG. 3A. The measured AOTF output of spectral profiles for the 8 excitation wavelengths used for the 10 fps live-cell demonstration are shown in FIG. 4A.

In this demonstration, the 8 channels of the RF synthesizer were preset to 8 different fixed RF frequencies and powers providing 8 fixed excitation wavelengths and intensities for the AOTF output. These 8 excitations were sequentially applied to each successive camera frame in cycles, so that full-frame excitation-spectral images were obtained every 8 consecutive camera frames.

For the 6-fluorophore target imaging of fixed cells, the preset AOTF driving frequencies were 61.567, 63.400, 65.148, 66.749, 68.454, 70.277, 71.500, and 82.370 MHz, corresponding to excitations centered at 565, 555, 545, 535, 525, 515, 505, and 460 nm, respectively as shown in FIG. 3A, and they were synchronized with 10 fps sCMOS recording. The expected recorded excitation spectra of the different fluorophores at the 8 excitation wavelengths in the fixed-cell obtained by multiplying the power-normalized spectral profiles of FIG. 3A is shown in FIG. 3B.

Similarly, for the 6-target imaging of live cells and all biosensor experiments, the 8 AOTF driving frequencies were 61.567, 63.400, 65.148, 66.995, 70.277, 72.700, 78.420, and 82.370 MHz, corresponding to excitations centered at 565, 555, 545, 530, 515, 500, 475, and 460 nm, respectively as shown in FIG. 4A, and these were also synchronized with fps sCMOS recording. The expected recorded excitation spectra of different fluorophores at the 8 excitation wavelengths for the live-cell demonstration is shown in FIG. 4B. Full width at half maximum (FWHM) of the AOTF output as a function of the central wavelength was also plotted.

For the fixed-cell demonstration, COS-7 cells were plated in an 8-well LabTek chamber at ˜30% confluency. After 24 h, cells were fixed using 3% paraformaldehyde and 0.1% glutaraldehyde in phosphate-buffered saline (PBS) followed by two washes with 0.1% sodium borohydride in PBS. Cells were blocked and permeabilized in a blocking buffer (3% bovine serum albumin with either 0.5% Triton X-100 or 0.02% saponin in PBS), followed by primary and secondary antibody labeling. Primary antibodies used were chicken anti-α-tubulin (ab89984, Abcam) for labeling of microtubules, rabbit anti-Nogo (ab47085, Abcam) for labeling of ER, mouse IgG1 anti-NPM1 (32-5200, ThermoFisher) for labeling of nucleoli, and mouse IgG2a anti-Tom20 (sc-17764, Santa Cruz Biotech) for labeling of mitochondria.

Secondary antibodies (Jackson ImmunoResearch) were labeled via reaction with dye NHS esters: CF514 (92103, Biotium), CF568 (92131, Biotium), ATTO 532 (AD532-31, ATTO-TEC) and ATTO 542 (AD542-31, ATTO-TEC). After antibody staining, cells were incubated in LipidSpot488 (300-1000×, 70065, Biotium) and SYBR Gold (300-1000×, S11494, ThermoFisher) in PBS to stain lipid droplets and nuclear DNA for 30 min, and the sample was washed with PBS three times for 10 min before imaging in PBS.

For the Live-cell demonstration, COS-7, U2-OS, and HeLa cells were cultured in Dulbecco's modified Eagle's medium (DMEM) (Gibco 31053-028) supplemented with 10% fetal bovine serum (Corning), 1× GlutaMAX Supplement, and 1× non-essential amino acids, at 37° C. and 5% CO². For live-cell experiments, cells were cultured in Lab-Tek 8-well chambered coverglass (ThermoFisher), and transiently transfected with the above plasmids, either alone or in combination. Transfection was performed using Lipofectamine 3000 (ThermoFisher) or the Neon Transfection System (ThermoFisher), following the manufacturers' instructions.

For live-cell staining of the plasma membrane, lipid droplet, and DNA, wheat germ agglutinin (WGA) CF532 (300-500×, 29064, Biotium), LipidSpot488 (300-1000×, 70065, Biotium), and SYBR Green (15000-100000×, S7536, ThermoFisher) were added to the medium for 30 min at 37° C. and washed 3 times with DMEM before imaging. Imaging buffer was the regular culture medium with the addition of 25 mM HEPES at pH 7.4 (15630106, Gibco) or a commercial buffer based on MOPS (Hibernate A, BrainBits), with similar results observed.

With this setup, images of fixed cells were acquired using an 8 wavelength excitation scheme and six labels. Six distinct subcellular structures were labeled with 6 fluorescent dyes (see FIG. 3B) that overlapped substantially in the excitation spectrum. Specifically, LipidSpot 488 labeled lipid droplets (LDs), SYBR Green labeled mitochondrial DNA, Mito-PhiYFP labeled mitochondrial matrix, WGA-CF532 labeled cell membrane, LysoBrite Orange labeled lysosomes, and tdTomato-ER3 labeled the ER.

Little crosstalk was observed between the 6 unmixed images with use of the 8-wavelength excitation scheme accomplished in 0.8 seconds (8 frames at 10 fps). However, the raw images at each wavelength contained substantial contributions from multiple fluorophores due to heavy spectral overlaps. Forcing the same 6-fluorophore unmixing procedure onto samples singly labeled by each fluorophore showed an average crosstalk of 1.3% between fluorophores. Simulations also showed that despite the non-ideal spectral profile of the AOTF output, low crosstalk was achievable at moderate noise levels for the 6 fluorophores of FIG. 3B and for the six live-cell fluorophores of FIG. 4B.

Next, the 8 excitation-wavelength spectral microscopy was applied to live cells. With a combination of fluorescent protein (FP) labels and live-cell stains, 6 distinct subcellular targets were labeled were well unmixed at an averaged 1.3% target-to-target crosstalk.

At a moderate camera framerate of 10 fps, a full-frame excitation-spectral image (and thus unmixed 6-target image) was obtained every 0.8 s. It was possible to visualize lysosome and endoplasmic reticulum (ER) both marked the fission site of a mitochondrion in live COS-7 cells. It was also possible to visualize mitochondrial DNA that was also divided in this process, consistent with previous, separate observations.

To demonstrate faster imaging, 4-wavelength excitation cycles were synchronized with a 240-fps camera framerate imaging 4 subcellular targets at a 16.7 ms time resolution. For the 4-excitation wavelength imaging of fast dynamics in live cells, 4 RF channels were set to 63.400, 70.277, 72.700, and 82.370 MHz, corresponding to excitations centered at 555, 515, 500, and 460 nm, respectively, and they were synchronized with 240 fps EMCCD or 202 fps sCMOS recordings.

This enabled the simultaneous detection of the fast structural changes of ER, mitochondria, mitochondrial DNA, and lipid droplets in the same field of view. In particular, the fast fusion of two lipid droplets was observed to occur within the 16.7 ms timeframe. A similar event was observed associated fast retraction of ER.

In another 4-wavelength performed at a 202-fps framerate (19.8 ms time resolution), demonstrated how an ER tubule first brought two lipid droplets closer in discrete, ˜0.1 s steps, and then mediated their fusion through fast ER reorganization, a process substantially different from existing models.

### Example 3

To further demonstrate the functionality of the system and methods, one embodiment of a quantitative imaging scheme was demonstrated using absolute pH imaging in live cells via unmixing and quantification of two different fluorophores.

One class of biosensors works on contrasting fluorescence properties between their analyte-bound and unbound states. The FP pureed is such a bi-state sensor that exhibits distinct excitation spectra upon protonation and deprotonation. Although this property has enabled ratiometric pH detection with two excitation wavelengths, absolute pH mapping is usually not performed.

Absolute pH mapping was demonstrated by quantifying the respective abundances of the protonated (HA) and deprotonated (A⁻) species of pHRed at each pixel. For pHRed, excitation spectra at different pH values were obtained from pHRed-expressing COS-7 cells that were proton-permeabilized with valinomycin and nigericin and immersed in a series of buffer standards (Intracellular pH Calibration Buffer Kit, P35379, ThermoFisher, plus additional homemade buffers).

Initially, the excitation spectra of pHRed in proton-permeabilized cells at low (4.5) and high (11) pH extremes, under which conditions the FP should be in the pure HA and A⁻ states, respectively were first obtained. The resultant abundances at each pH level fitted well to the simple Henderson-Hasselbalch equation based on thermodynamic equilibrium:

$$pH = {pH}_{0} + log \frac{[ {A}^{-} ]}{[ HA ]}$$

in which pH₀ is the only fitting parameter, and [A⁻] and [HA] are the abundances of the two states. From the fit, a pH₀=7.87 was obtained, a value that is close to the apparent pKₐ of pHRed (˜7.8).

This good fit to theory allowed imaging of absolute pH in live cells by first unmixing the excitation spectrum at every pixel and then feeding the resultant HA and A⁻ abundances into the Henderson-Hasselbalch equation. Besides achieving high confidences for HA and A⁻ abundances and hence pH, this approach enabled us to readily incorporate additional fluorophores, as discussed below.

For pHRed expressed in the cytoplasm, a homogenous pH of between about 7.2 and 7.4 was observed in common cell lines. By comparison, Mito-pHRed in the mitochondrial matrix showed typical pH of between about 7.7 and 8.1 in live cells while exhibiting sporadic, spontaneous jumps. Treating the cells with the uncoupler carbonyl cyanide m-chlorophenyl hydrazone (CCCP) led to fast drops of the mitochondrial matrix pH to between about 7.4 to 7.5. While these results are qualitatively consistent with previous observations, the full-frame fast recording quantified absolute pH at high spatiotemporal resolutions.

For the CCCP-treated cells, diverse pH dynamics between different mitochondria in the same cell was observed. For the spontaneous pH jumps in untreated live cells, it was shown that the matrix pH of individual mitochondria abruptly rose by about 0.1 to 0.3 units in about 2 s. Such fast processes often coincided with abrupt mitochondrial shape changes that were fittingly captured with the 0.8 s full-frame time resolution scheme. The pH then gradually decreased in approximately 30 s and overshot to a value about 0.05 lower than the starting pH, a process often accompanied by further changes in mitochondrial shape.

### Example 4

To illustrate multiplexing of the quantitative biosensing and multi-fluorophore imaging capabilities, absolute pH imaging was combined with three additional fluorophore labels to elucidate the Parkin-mediated mitophagy pathway, an interesting multistate system. For live HeLa cells lacking Parkin, absolute pH imaging with Mito-pHRed showed that after about 30 minutes of CCCP treatment, the mitochondrial matrix pH gradually recovered from the initial drop to reach approximately 7.7 at around 1 hour. Relatively homogenous pH values were detected between different mitochondria in the same cell, although some cell-to-cell variations were noticed. Remarkably, under the same experimental conditions, HeLa cells expressing fluorescently untagged Parkin exhibited a large heterogeneity in mitochondrial matrix pH (˜6.7-7.7), with notably dissimilar pH values observed for neighboring mitochondria in the same cell.

To examine whether these different mitochondrial pH states could correspond to different stages of mitophagy, three FP-tagged mitophagy-stage markers, mOrange2-Parkin, PhiYFP-LC3, and LAMP1-Clover, were co-expressed in the cell and similar long-term CCCP treatments were performed. The five fluorescent species (A⁻ and HA forms of pHRed, plus mOrange2, PhiYFP, and Clover were well unmixed with the 8-wavelength excitation spectral microscopy. Close evaluation of the Mito-pHRed-quantified pH values at the mitochondria that were enclosed by the three fluorophore-tagged markers showed that the Parkin-marked mitochondria, at the onset of mitophagy, had a matrix pH of between about 7.7 and 7.8. These pH values are comparable to the high-pH population in the Parkin-expressing cells without fluorophore-tagged markers.

By comparison, mitochondria positive for both Parkin and the autophagosome marker LC3 exhibited lower matrix pH values of between about 7.5 and 7.7. Mitochondria marked by LC3 alone showed yet lower matrix pHs of between about 7.3 and 7.6. The lower values in this distribution matched cytoplasmic pH, suggesting complete loss of mitochondrial function. Lastly, mitochondria enclosed by the lysosome marker LAMP1 showed the lowest pHs (<˜7.4), suggesting a gradual acidification of the mitochondrial matrix after lysosome fusion.

To further validate these results, the fluorophores tagged to the above three mitophagy-stage markers were permutated. The resulting spectrally resolved images of Mito-pHRed, PhiYFP-Parkin, Clover-LC3, and LAMP1-mOrange2 showed comparable matrix pH trends for mitochondria positive for the different stage markers. A model in which the mitochondrial matrix pH progressively decreases along the mitophagy pathway is suggested. These results contrast with previous efforts employing pH-sensitive FPs to detect mitophagy, in which only the mitophagy end results are detected and the absolute pH is not measured to elucidate the pathway. Moreover, with the fast full-frame imaging capability, the LAMP1-enclosed mitochondria, with their characteristic low matrix pH, moved much faster when compared to their Parkin- and LC3-enclosed counterparts, reflecting the high mobility of lysosomes in cells.

### Example 5

FRET imaging in live cells via resolving the excitation spectrum was used to further illustrate the adaptability of the systems and methods. Another major class of biosensors is based on Förster resonance energy transfer (FRET). Although modern FRET imaging often focuses on separating the fluorescence emissions of the donor and acceptor fluorophores, early FRET spectroscopy studies favored the analysis of the excitation spectrum. When monitoring the emission from the acceptor, FRET gives rise to new features in the excitation spectrum corresponding to donor absorption.

To demonstrate a microscopy version of this spectroscopy approach, the Clover-mRuby2 fluorophore pair was used. Since the emission band well-matched the emission of mRuby2 but not Clover, for COS-7 cells co-expressing the two free FPs, the recorded 8-wavelength excitation spectrum was dominated by mRuby2. Unmixing this spectrum into mRuby2 and Clover components showed the peak height of the latter to be approximately 7% of the former.

In contrast, for a construct in which Clover and mRuby2 were directly linked, the excitation spectrum showed a substantial rise at about 500 nm that was attributable to FRET-induced mRuby2 emission due to Clover absorption. Unmixing the spectrum into mRuby2 and Clover components showed the peak height of the latter to be about 62% of the former. Subtracting the above 7% bleed-through from Clover emission and considering the near-identical peak extinction coefficients of mRuby2 and Clover gave an estimated FRET efficiency of 0.55, in agreement with emission spectroscopy results on a similar construct (0.55).

To demonstrate the system and methods further, a FRET sensor for quantifying macromolecular crowding in live cells by linking Clover and mRuby2 with a long, conformationally flexible domain was constructed and tested. When expressed in the COS-7 cell cytoplasm, this sensor exhibited lower FRET efficiencies than the directly linked Clover-mRuby2, consistent with increased donor-acceptor distances. Upon treating the live cells with 150% and 50% osmotic pressures of the normal medium, the measured FRET efficiency rose and dropped substantially, as expected for the reduced and increased donor-acceptor distances under raised and lowered levels of macromolecular crowding inside the cells, respectively.

Through continuous recording under the 8-wavelength excitation scheme at 10 fps and performing the above FRET analysis for every pixel, full-frame FRET images at 0.8 s time resolution were obtained. The FRET images unveiled rich spatiotemporal heterogeneities in macromolecular crowding in live cells. In untreated COS-7 cells, the observed FRET efficiency was largely uniform within each cell but varied substantially (˜0.29-0.33) between cells.

Interestingly, it was shown that the 150% hypertonic treatment resulted in quick rises in the intracellular FRET signal that plateaued in approximately 25 seconds compared to the 50% hypotonic treatment that led to slow decreases in the FRET signal over approximately 500 seconds and subsequent recoveries. These results may be rationalized with the contrasting, quick reduction versus slow expansion-recovery of cell volumes under hypertonic and hypotonic conditions. At the subcellular level, it was found that for the hypertonic treatment, the nuclear FRET signals rose slower and achieved lower final increases when compared to the cytoplasm (˜0.03 vs. ˜0.05). This result may be construed as that the nuclear envelop retarded volume reduction and thus a rise in macromolecular crowding. In contrast, for the opposite, slow hypotonic process, the cytoplasmic FRET signals dropped slower than the nuclei, and for some cells showed complete recovery over long time. This result may be attributed to the regulatory volume decrease processes in the cytoplasm.

It will be appreciated that this disclosure describes systems and methods for excitation spectral microscopy using frame-synchronized acousto-optic scanning of fluorescent excitation wavelengths. In one embodiment, the excitation is switched between preset wavelength profiles of selected band width, intensity and wavelengths in each successive frame to produce full frame excitation spectral images. Linear unmixing of the images of targets of components that are individually labeled with different fluorophores can be simultaneously imaged with high temporal resolution and low crosstalk and the local abundance of each fluorophore at each pixel can be quantified. Fluorophore decomposed micrographs of the sample can be obtained by rendering the abundance in each pixel as an image.

From the description herein, it will be appreciated that the present disclosure encompasses multiple embodiments which include, but are not limited to, the following:

A method for excitation spectral microscopy, the method comprising: (a) providing one or more subjects with a plurality of components for imaging; (b) labeling one or more components of the subject with a unique fluorophore label; and (c) selectively exciting each fluorophore with an excitation beam synchronously with an acquired frame of an imaging device; (d) switching excitation beam wavelength with each successive frame of the imaging device; (e) quantifying a local abundance of each unique fluorophore at each pixel of the frame; and (f) rendering a fluorophore-decomposed micrograph of the labeled subject from the quantified abundance of each fluorophore.

The method of any preceding or following implementation, wherein the subject comprises one or more fixed cells or one or more living cells.

The method of any preceding or following implementation, wherein the excitation beam comprises a beam profile of a bandwidth, intensity and range of one or more wavelengths that are optimized for exciting a unique fluorescent label.

The method of any preceding or following implementation, wherein the fluorescent label is at least one member of the group consisting of LipidSpot 488, SYBR Gold, CF514, ATTO 532, ATTO 542 and CF568.

The method of any preceding or following implementation, wherein the fluorescent label is at least one member of the group consisting of SYBR Green, Mito-PhiYFP, WGA-CF532, LysoBrite Orange, and tdTomato-ER3.

The method of any preceding or following implementation, wherein quantification of the local abundance of each fluorophore comprises: measuring excitation spectra of one or more fluorophore labels from a singly labeled sample; and linearly unmixing an excitation spectrum of every pixel based on the excitation spectrum of each fluorophore using singly labeled samples.

An imaging method, comprising simultaneously imaging a plurality of subcellular targets, labeled by common fluorophores of substantial spectral overlap, in live cells in the wide-field at low crosstalk and high spatiotemporal resolutions using a standard epifluorescence microscope with a single emission band, through frame-synchronized fast scanning of the excitation wavelength from a white lamp at about 10 nm resolution.

An imaging method, comprising simultaneously imaging a plurality of subcellular targets and quantitating intracellular biochemical parameters, labeled by common fluorophores and biosensors of substantial spectral overlap, in live cells at low crosstalk and high temporal resolutions, using a single, fixed fluorescence emission detection band, through frame-synchronized fast scanning of the excitation wavelength from a white lamp via an acousto-optic tunable filter (AOTF).

The method of any preceding or following implementation, wherein concurrent imaging of multiple subcellular targets and biosensors comprises up to six targets, the crosstalk comprises about one percent and the temporal resolution is as low as about ten milliseconds.

An apparatus for excitation spectral microscopy, comprising: (a) an epifluorescence microscope with an excitation beam input and an image output; (b) a multispectral illumination source operably coupled to the excitation beam input of the epifluorescence microscope, configured to produce excitation beams with controlled wavelengths; (c) an imaging device coupled to the image output of the epifluorescence microscope, the imaging device capturing image output frame by frame with a controllable frame rate; (d) one or more processors operably connected to the multispectral illumination source and to the imaging device; and (e) a non-transitory memory storing executable instructions that, if executed by the one or more processors, configure the apparatus to: (i) control wavelength range of the excitation beam; (ii) synchronize actuation of the excitation beam with capture of the image output by the imaging device; and (iii) analyze captured excitation spectral images.

The apparatus of any preceding or following implementation, wherein the instructions when executed by the processor further perform steps comprising creating one or more preset excitation profiles of a bandwidth, an intensity and a wavelength; and switching the excitation between a plurality of preset wavelength profiles in successive image frames.

The apparatus of any preceding or following implementation, wherein the excitation profile comprises: an excitation beam profile of a bandwidth, intensity and range of one or more wavelengths that are optimized for exciting a specific fluorescent label.

The apparatus of any preceding or following implementation, wherein the instructions when executed by the processor further perform steps comprising controlling a frame rate of the imaging device.

The apparatus of any preceding or following implementation, wherein the instructions when executed by the processor further perform steps comprising quantifying a local abundance of a fluorescence of a fluorophore at each pixel of a frame; and rendering a fluorophore-decomposed micrograph of a labeled subject from the quantified abundance of the fluorophore.

The apparatus of any preceding or following implementation, wherein the multispectral illumination source comprises: a white light source; a polarizer positioned downstream of the light source; an acousto-optic tunable filter (AOTF) positioned downstream of the polarizer for receiving a polarized beam from the light source; and a second polarizer positioned downstream of the AOTF for receiving a polarized excitation beam from the AOTF, the second polarizer downstream of the excitation beam input of the epifluorescence microscope.

The apparatus of any preceding or following implementation, wherein the epifluorescence microscope further comprises a single band filter cube.

The apparatus of any preceding or following implementation, wherein the epifluorescence microscope further comprises an objective lens with a back focal plane positioned for receiving an excitation beam from the second polarizer.

The apparatus of any preceding or following implementation, wherein the imaging device further comprises: an RF synthesizer coupled to imaging device and to the AOTF and configured to select one or more excitation wavelengths for scanning; and wherein the imaging device and processor are configured to control the RF synthesizer to apply an RF frequency to the AOTF on a frame-by-frame basis; and wherein the RF synthesizer is configured for synchronized control of the excitation wavelength for each image frame.

A system for excitation spectral imaging, the system comprising: (a) an excitation light source configured to produce an excitation beam with a controlled wavelength, intensity and bandwidth; (b) an epifluorescence microscope with an objective coupled to the excitation light source configured to illuminate a target field with an excitation beam; and (c) an imaging device with a controller operably coupled to the excitation light source, the imaging device configured for frame-by-frame imaging of the target field at a different excitation wavelength for each frame.

The system of any preceding or following implementation, wherein the excitation light source comprises: a white light source; a polarizer positioned downstream of the white light source; an acousto-optic tunable filter (AOTF) positioned downstream of the polarizer for receiving a polarized beam from the white light source; a second polarizer positioned downstream of the AOTF for receiving a polarized excitation beam from the AOTF, the second polarizer downstream of an excitation beam input of the epifluorescence microscope; and a single bandpass filter positioned downstream of an output of the epifluorescence microscope providing the excitation beam.

The system of any preceding or following implementation, wherein the imaging device with a controller comprises: an RF synthesizer coupled to the AOTF and imaging device controller; wherein the image capture device controller is configured to control the RF synthesizer to apply an RF frequency to the AOTF on a frame-by-frame basis; and wherein the RF synthesizer is configured for synchronized control of the excitation wavelength, intensity, and bandwidth for each image frame.

The system of any preceding or following implementation, wherein the imaging device controller is configured to control the RF synthesizer for frame-by-frame imaging at a different excitation wavelength for each frame according to one of a plurality of fluorophore excitation profiles of a bandwidth, intensity and range of one or more wavelengths that are optimized for exciting a specific fluorescent label.

The system of any preceding or following implementation, wherein the imaging device controller is further configured to control a frame rate of image acquisition of the imaging device.

As used herein, term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system or method.

As used herein, the term “set” refers to a collection of one or more objects. Thus, for example, a set of objects can include a single object or multiple objects.

Relational terms such as first and second, top and bottom, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to +10°, such as less than or equal to 5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential features or elements of the technology describes herein or any or all the claims.

In addition, in the foregoing disclosure various features may grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after that application is filed. Accordingly the reader should consult the application as filed for the original content of the disclosure. Any deletion of content of the disclosure should not be construed as a disclaimer, forfeiture or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A method for excitation spectral microscopy, the method comprising:
(a) providing one or more subjects with a plurality of components for imaging;
(b) labeling one or more components of the subject with a unique fluorophore label;
(c) selectively exciting each fluorophore with an excitation beam synchronously with an acquired frame of an imaging device;
(d) switching excitation beam wavelength with each successive frame of said imaging device;
(e) quantifying a local abundance of each unique fluorophore at each pixel of the frame; and
(f) rendering a fluorophore-decomposed micrograph of the labeled subject from the quantified abundance of each fluorophore.

2. The method of claim 1, wherein said subject comprises one or more fixed cells or one or more living cells.

3. The method of claim 1, wherein said excitation beam comprises a beam profile of a bandwidth, intensity and range of one or more wavelengths that are optimized for exciting a specific fluorescent label.

4. The method of claim 1, wherein said fluorescent label is at least one member of the group consisting of LipidSpot 488, SYBR Gold, CF514, ATTO 532, ATTO 542 and CF568.

5. The method of claim 1, wherein said fluorescent label is at least one member of the group consisting of SYBR Green, Mito-PhiYFP, WGA-CF532, LysoBrite Orange, and tdTomato-ER3.

6. The method of claim 1, wherein quantification of the local abundance of each fluorophore comprises:
measuring excitation spectra of one or more fluorophore labels from a singly labeled sample; and
linearly unmixing an excitation spectrum of every pixel based on the excitation spectrum of each fluorophore using singly labeled samples.

7. An apparatus for excitation spectral microscopy, comprising:
(a) an epifluorescence microscope with an excitation beam input and an image output;
(b) a multispectral illumination source operably coupled to the excitation beam input of the epifluorescence microscope, configured to produce excitation beams with controlled wavelengths;
(c) an imaging device coupled to the image output of the epifluorescence microscope, said imaging device capturing image output frame by frame with a controllable frame rate;
(d) one or more processors operably connected to the multispectral illumination source and to the imaging device; and
(e) a non-transitory memory storing executable instructions that, if executed by the one or more processors, configure the apparatus to:
(i) control wavelength range of the excitation beam;
(ii) synchronize actuation of the excitation beam with capture of the image output by the imaging device; and
(iii) analyze captured excitation spectral images.

8. The apparatus of claim 7, wherein said instructions when executed by the processor further perform steps comprising:
creating one or more preset excitation profiles of a bandwidth, an intensity and a wavelength; and
switching the excitation between a plurality of preset wavelength profiles in successive image frames.

9. The apparatus of claim 8, wherein said excitation profile comprises:
an excitation beam profile of a bandwidth, intensity and range of one or more wavelengths that are optimized for exciting a specific fluorescent label.

10. The apparatus of claim 7, wherein said instructions when executed by the processor further perform steps comprising:
controlling a frame rate of the imaging device.

11. The apparatus of claim 7, wherein said instructions when executed by the processor further perform steps comprising:
quantifying a local abundance of a fluorescence of a fluorophore at each pixel of a frame; and
rendering a fluorophore-decomposed micrograph of a labeled subject from the quantified abundance of the fluorophore.

12. The apparatus of claim 7, wherein said multispectral illumination source comprises:
a white light source;
a polarizer positioned downstream of the light source;
an acousto-optic tunable filter (AOTF) positioned downstream of the polarizer for receiving a polarized beam from the light source; and
a second polarizer positioned downstream of the AOTF for receiving a polarized excitation beam from the AOTF, the second polarizer downstream of the excitation beam input of the epifluorescence microscope.

13. The apparatus of claim 7, wherein said epifluorescence microscope further comprises a single band filter cube.

14. The apparatus of claim 7, wherein said epifluorescence microscope further comprises an objective lens with a back focal plane positioned for receiving an excitation beam from the second polarizer.

15. The apparatus of claim 7, wherein said imaging device further comprises:
an RF synthesizer coupled to imaging device and to the AOTF and configured to select one or more excitation wavelengths for scanning; and
wherein the imaging device and processor are configured to control the RF synthesizer to apply an RF frequency to the AOTF on a frame-by-frame basis; and
wherein said RF synthesizer is configured for synchronized control of the excitation wavelength for each image frame.

16. A system for excitation spectral imaging, the system comprising:
(a) an excitation light source configured to produce an excitation beam with a controlled wavelength, intensity and bandwidth;
(b) an epifluorescence microscope with an objective coupled to the excitation light source configured to illuminate a target field with an excitation beam; and
(c) an imaging device with a controller operably coupled to the excitation light source, the imaging device configured for frame-by-frame imaging of the target field at a different excitation wavelength for each frame.

17. The system of claim 16, wherein said excitation light source comprises:
a white light source;
a polarizer positioned downstream of the white light source;
an acousto-optic tunable filter (AOTF) positioned downstream of the polarizer for receiving a polarized beam from the white light source;
a second polarizer positioned downstream of the AOTF for receiving a polarized excitation beam from the AOTF, the second polarizer downstream of an excitation beam input of the epifluorescence microscope; and
a single bandpass filter positioned downstream of an output of the epifluorescence microscope providing said excitation beam.

18. The system of claim 16, wherein said imaging device with a controller comprises:
an RF synthesizer coupled to the AOTF and imaging device controller;
wherein the image capture device controller is configured to control the RF synthesizer to apply an RF frequency to the AOTF on a frame-by-frame basis; and
wherein said RF synthesizer is configured for synchronized control of the excitation wavelength, intensity, and bandwidth for each image frame.

19. The system of claim 18, wherein said imaging device controller is configured to control the RF synthesizer for frame-by-frame imaging at a different excitation wavelength for each frame according to one of a plurality of fluorophore excitation profiles of a bandwidth, intensity and range of one or more wavelengths that are optimized for exciting a specific fluorescent label.

20. The system of claim 18, wherein said imaging device controller is further configured to control a frame rate of image acquisition of the imaging device.
